(19) United States
(12) Patent Application Publication
(10) Pub. No.: US 2021/0282758 A1
(43) Pub. Date: Sep. 16, 2021

(54) TISSUE SPECIMEN BAGS AND SYSTEMS

(71) Applicant: Covidien LP, Mansfield, MA (US)

(72) Inventors: George MATTA, Plainville, MA (US); Jacob C. Baril, Norwalk, CT (US); Saumya Banerjee, Hamden, CT (US); Christopher M. Meehan, New Haven, CT (US); Amy Kung, Hamden, CT (US); Thomas A. Zammataro, North Haven, CT (US); Scott J. Prior, Shelton, CT (US)

(21) Appl. No.: 16/817,018

(22) Filed: Mar. 12, 2020

(51) Int. Cl. A61B 17/00

(52) U.S. Cl. CPC A61B 17/00234; A61B 2017/00287; A61B 2017/00862; A61B 2017/00902; A61B 2017/00955; A61B 2017/00867

(57) ABSTRACT

A tissue specimen bag includes a bag body and a support ring. The bag body has a closed end and an open end defining a mouth, and defines a channel circumferentially surrounding the mouth. The bag body further includes a plurality of cut-outs defined within the bag body and spaced-apart circumferentially about the mouth such that the channel is discontinuous. The support ring extends through the channel, is resiliently flexible, and is configured to bias the mouth towards an open condition. Portions of the support ring are exposed within the plurality of cut-outs to enable direct interfacing with the support ring without contacting the bag body. A tissue specimen retrieval system includes the tissue specimen bag and a grasper configured to grasp one of the exposed portions of the support ring without contacting the bag body to facilitate manipulation of the tissue specimen bag.

### BACKGROUND

### Technical Field

The present disclosure relates to tissue specimen retrieval from an internal body cavity and, more particularly, to tissue specimen bags and systems facilitating retrieval of a tissue specimen from an internal body cavity.

### Background of Related Art

In minimally-invasive surgical procedures, operations are carried out within an internal body cavity through small entrance openings in the body. The entrance openings may be natural passageways of the body or may be surgically created, for example, by making a small incision into which an access device is inserted.

Minimally-invasive surgical procedures may be used for partial or total retrieval of a tissue specimen from an internal body cavity. However, the restricted access provided by minimally-invasive openings (natural passageways and/or surgically created openings) presents challenges with respect to maneuverability and visualization. The restricted access also presents challenges when the tissue specimen is required to be removed. As such, a tissue specimen that is deemed too large for intact retrieval may be broken down into a plurality of smaller pieces to facilitate retrieval from the internal body cavity.

### SUMMARY

As used herein, the term “distal” refers to the portion that is described which is further from a user, while the term “proximal” refers to the portion that is being described which is closer to a user. The terms “substantially” and “approximately,” as utilized herein, account for industry-accepted material, manufacturing, measurement, use, and/or environmental tolerances. Further, any or all of the aspects and features described herein, to the extent consistent, may be used in conjunction with any or all of the other aspects and features described herein.

Provided in accordance with aspects of the present disclosure is a tissue specimen bag including a bag body and a support ring. The bag body has a closed end and an open end defining a mouth. The bag body defines a channel circumferentially surrounding the mouth and includes a plurality of cut-outs defined within the bag body and spaced-apart circumferentially about the mouth such that the channel is discontinuous. The support ring extends through the channel. The support ring is resiliently flexible and configured to bias the mouth towards an open condition. Portions of the support ring are exposed within the plurality of cut-outs to enable direct interfacing with the support ring without contacting the bag body.

In an aspect of the present disclosure, the support ring is formed from nitinol.

In another aspect of the present disclosure, the bag body is formed from a transparent material.

In still another aspect of the present disclosure, the bag body is formed from a thermoplastic polyurethane.

In yet another aspect of the present disclosure, the support ring is formed as a continuous ring.

In still yet another aspect of the present disclosure, the support ring includes at least one segment. End portions of the at least one segment are coupled to one another to form the support ring. In such aspects, a connector may connect the end portions with one another. The connector may be flexible.

A tissue specimen retrieval system provided in accordance with the present disclosure includes the tissue specimen bag according to any of the above-detailed aspects of other aspects provided herein, and a grasper. The grasper is configured to grasp one of the exposed portions of the support ring without contacting the bag body to facilitate manipulation of the tissue specimen bag.

In aspects, the grasper includes first and second jaw members. At least one of the first or second jaw members is movable relative to the other from a spaced-apart position to an approximated position to grasp the exposed portions.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a side view of a tissue specimen bag provided in accordance with aspects of the present disclosure;

FIG. 2 is an enlarged, side view of the area of detail indicated as “2” in FIG. 1;

FIG. 3 is an enlarged, perspective view illustrating a connector connecting ends of the support ring of the tissue specimen bag of FIG. 1; and

FIG. 4 is an enlarged, perspective view of a portion of the tissue specimen bag of FIG. 1 including a grasper engaged thereon to facilitate manipulation of the bag.

### DETAILED DESCRIPTION

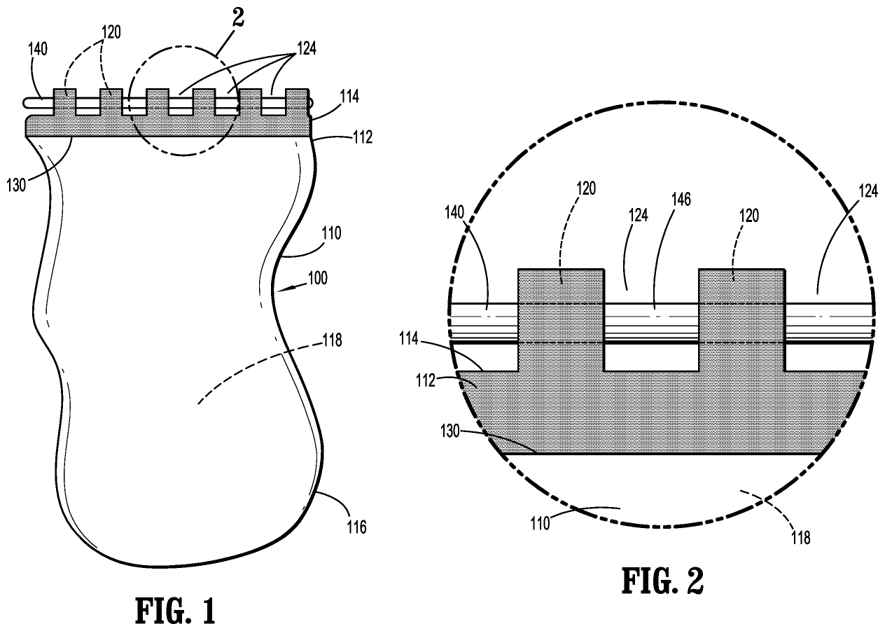

Turning to FIGS. 1 and 2, a tissue specimen bag provided in accordance with the present disclosure is shown generally identified by reference numeral 100. Tissue specimen bag 100 includes a bag body 110 and a support ring 140 disposed at the open end 112 of bag body 110 to define and maintain a mouth 114 in an open condition at open end 112 of bag body 110.

Bag body 110 of specimen bag 100 includes open end 112, an opposing closed end 116, and an internal chamber 118. Bag body 110 may be formed from any suitable flexible bio-compatible material (or materials), e.g., a thermoplastic polyurethane material, and may be transparent to enable visualization into internal chamber 118 and through bag body 110.

Open end 112 of bag body 110 defines a channel 120 surrounding mouth 114 and a plurality of spaced-apart cut-outs 124 disposed about the circumference of mouth 114. Channel 120 may be formed by folding over the edge of bag body 110 at open end 112 back onto bag body 110 and attaching the edge or a portion of bag body 110 adjacent the edge back onto bag body 110 along a seam 130, e.g., via welding, heat sealing, adhesive, or in any other suitable manner. Spaced-apart cut-outs 124 may then be defined within, e.g., material is cut out from, the folded portion of bag body 110 such that channel 120 is discontinuously defined about mouth 114 and such that cut-outs 124 are circumferentially-spaced about mouth 114. Other suitable manufacturing methods are also contemplated.

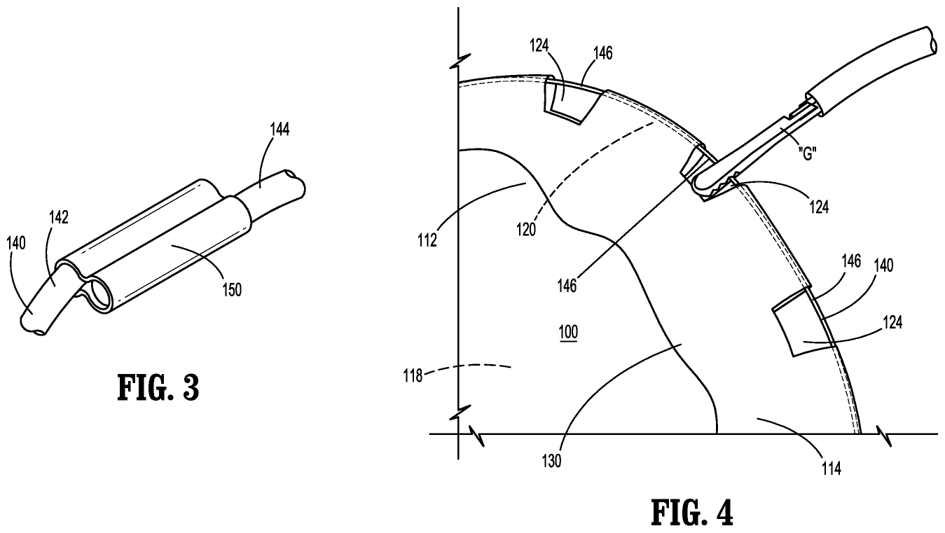

Support ring 140 is routed through channel 120 such that support ring 140 is exposed within each cut-out 124. Support ring 140 may be formed from a resiliently flexible material, e.g., nitinol, spring steel, or other suitable material, that is biased towards a ring-shaped configuration and is sufficiently rigid to maintain mouth 114 in an open condition to enable insertion and withdrawal of tissue into and from internal chamber 118. The material forming support ring 140 is also sufficiently flexible to enable collapse to facilitate insertion through an opening in tissue, access port, a trocar, etc., and/or loading into a deployment device. Alternatively or additionally, support ring 140 may include one or more ring segments interconnected via one or more flexible connectors, e.g., elastomeric joints, springs, reduced-thickness materials, hinges, pivots, etc., such that in addition or as an alternative to the segment(s) of support ring 140 flexing themselves, the flexible connector(s) may flex to facilitate collapse of support ring 140. Connector 150 (FIG. 3), detailed below, may be a rigid connector, or may be a flexible connector such as noted above.

Specimen bag 100 may be configured as a stand-alone bag that is collapsed, e.g., bent, rolled, folded, etc., for insertion into an internal surgical site, or may be loaded into a deployment device and deployed from the deployment device into the internal surgical site. Support ring 140 may be formed as a continuous, closed ring that is positioned adjacent the edge of bag body 110 prior to folding and attaching the edge or the portion of bag body 110 adjacent the edge back onto bag body 110 along the seam 130 such that, upon forming channel 120, support ring 140 is disposed within channel 120. Alternatively, support ring 140 may be formed as one or more segments that are first routed through the formed channel 120 and cut-outs 124 of bag body 110 about the circumference of mouth 114 and then i coupled to form support ring 140. With additional reference to FIG. 3, a connector 150 may be utilized to couple end portions 142, 144 of the one or more segments of support ring 140 with one another to form the closed support ring 140. Additional connectors 150 of any suitable configuration may be utilized where plural segments of support ring 140 are coupled to define the closed support ring 140. In other embodiments, support ring 140 is not closed but defines an open or discontinuous configuration.

Referring to FIG. 4, with support ring 140 extending through channel 120 circumferentially about mouth 114 of bag body 110 of specimen bag 100, the portions of support ring 140 extending through cut-outs 124 are exposed. In other words, cut-outs 124 provide access to exposed portions 146 of support ring 140. These exposed portions 146 of support ring 140 enable a grasper “G” or other suitable surgical tool, e., a surgical hook, to directly engage, e.g., grasp, or otherwise directly interface with support ring 140 without contacting bag body 110. The grasper “G” may include first and second jaw members at least one of which is movable relative to the other between spaced-apart and approximated positions for grasping objects, e.g., exposed portions 146 of support ring 140. Whether by grasper “G” or other suitable surgical tool, specimen bag 100 can be manipulated without damaging, e.g., ripping, bag body 110, and with greater control (by allowing direct grasping of a more-rigid structure (exposed portions 146 of support ring 140)).

From the foregoing and with reference to the various drawings, those skilled in the art will appreciate that certain modifications can be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A tissue specimen bag, comprising:
a bag body having a closed end and an open end defining a mouth, the bag body defining a channel circumferentially surrounding the mouth and including a plurality of cut-outs defined within the bag body and spaced-apart circumferentially about the mouth such that the channel is discontinuous; and
a support ring extending through the channel, wherein the support ring is resiliently flexible and configured to bias the mouth towards an open condition, wherein portions of the support ring are exposed within the plurality of cut-outs to enable direct interfacing with the support ring without contacting the bag body.

2. The tissue specimen bag according to claim 1, wherein the support ring is formed from nitinol.

3. The tissue specimen bag according to claim 1, wherein the bag body is formed from a transparent material.

4. The tissue specimen bag according to claim 1, wherein the bag body is formed from a thermoplastic polyurethane.

5. The tissue specimen bag according to claim 1, wherein the support ring is formed as a continuous ring.

6. The tissue specimen bag according to claim 1, wherein the support ring includes at least one segment, wherein end portions of the at least one segment are coupled to one another to form the support ring.

7. The tissue specimen bag according to claim 6, wherein a connector connects the end portions with one another.

8. The tissue specimen bag according to claim 7, wherein the connector is flexible.

9. A tissue specimen retrieval system, comprising:
a tissue specimen bag, including:
a bag body having a closed end and an open end defining a mouth, the bag body defining a channel circumferentially surrounding the mouth and including a plurality of cut-outs defined within the bag body and spaced-apart circumferentially about the mouth such that the channel is discontinuous; and
a support ring extending through the channel, wherein the support ring is resiliently flexible and configured to bias the mouth towards an open condition, wherein portions of the support ring are exposed within the plurality of cut-outs; and
a grasper configured to grasp one of the exposed portions of the support ring without contacting the bag body to facilitate manipulation of the tissue specimen bag.

10. The tissue specimen retrieval system according to claim 9, wherein the support ring is formed from nitinol.

11. The tissue specimen retrieval system according to claim 9, wherein the bag body is formed from a transparent material.

12. The tissue specimen retrieval system according to claim 9, wherein the bag body is formed from a thermoplastic polyurethane.

13. The tissue specimen retrieval system according to claim 9, wherein the support ring is formed as a continuous ring.

14. The tissue specimen retrieval system according to claim 9, wherein the support ring includes at least one segment, wherein end portions of the at least one segment are coupled to one another to form the support ring.

15. The tissue specimen retrieval system according to claim 14, wherein a connector connects the end portions with one another.

16. The tissue specimen retrieval system according to claim 15, wherein the connector is flexible.

17. The tissue specimen retrieval system according to claim 9, wherein the grasper includes first and second jaw members, at least one of the first or second jaw members movable relative to the other from a spaced-apart position to an approximated position to grasp the exposed portions.
